(19) United States
(12) Patent Application Publication
(10) Pub. No.: US 2017/0112589 A1
(43) Pub. Date: Apr. 27, 2017

(54) Distal Tip Soak Basin, Agitator and Methods of Use

(71) Applicant: Ryan Ramkhelawan, Collierville, TN (US)

(72) Inventor: Ryan Ramkhelawan, Collierville, TN (US)

(21) Appl. No.: 15/335,609

(22) Filed: Oct. 27, 2016

Related U.S. Application Data
(60) Provisional application No. 62/246,943, filed on Oct. 27, 2015.

(51) Int. Cl. A61B 90/70; B08B 9/032; A61B 1/12

(52) U.S. Cl. CPC A61B 90/70; A61B 1/122; B08B 9/0321; A61B 2090/701

(57) ABSTRACT

A distal tip soak basin, agitator, and methods of use, by providing a tube or sleeve with a closed end and an open end, an end cap having an aperture therein to retain a medical device, a perforated instrument holder or seal to seal the end cap around a medical device, a vibrating motor and hosing with friction fit sleeve, a battery, and an on/off switch, and, thus, functions to contain the distal tip and orifice of an endoscope in an enzymatic fluid for the purpose of pre-soaking and agitating the cleaning or soaking fluid, such as an enzymatic fluid, in the distal tip to delay the drying of body fluids and particulate contained inside the distal tip and orifice and/or internal channels.

### CROSS REFERENCE TO RELATED APPLICATIONS

To the full extent permitted by law, the present United States Non-provisional patent application hereby claims priority to and the full benefit of, United States Provisional Application entitled “Endoscopic Cleaning and Protection device,” having assigned Ser. No. 62/246,943, filed on Oct. 27, 2015, which is incorporated herein by reference in its entirety.

### TECHNICAL FIELD

The disclosure relates generally to medical pre-cleaning devices and more specifically it relates to medical pre-cleaning devices attached to distal end of endoscopes.

### BACKGROUND

An endoscope is a medical device used to look inside a body cavity or organ. The scope is inserted through a natural opening, such as the mouth or the rectum to perform a medical procedure called an endoscopy. Endoscopes may include a flexible insertion tube with one or more channels therein having a distal end and orifice or opening, housing a light delivery system to illuminate the organ or object under inspection, a camera to transmit images to a screen for image review and capture, and additional channel(s) to allow entry of medical instruments or manipulators, delivery of air or water, suction, biopsy retrieval and the like. Between uses from one patient to the next the endoscope, especially its internal channels must be cleaned of body fluids and particulate, and sterilized.

Disinfection is difficult due to the miniature design of the endoscope, hidden and/or retractable instruments, small orifices, and long narrow channels or passageways. To prevent body fluids and particulate from drying in these remote locations some pre-cleaning approaches have been utilized.

One previous pre-cleaning approach includes inserting the distal end and orifice into pre-soaked sponges with enzymatic fluid for the purpose of pre-soaking distal tip to delay the drying of body fluids and particulate contained inside the internal channels. One disadvantage of this approach is that this prior product does not include the ability to agitate or circulate the enzymatic pre-soak fluid contained in the distal tip or internal channels of the endoscope.

Another previous pre-cleaning approach includes inserting the distal end and orifice into a small plastic tube with a sealed end and a cap with enzymatic fluid for the purpose of pre-soaking distal tip to delay the drying of body fluids and particulate contained inside the internal channels. Again one disadvantage of this approach is that this prior product does not include the ability to agitate or circulate the enzymatic pre-soak fluid contained in the distal tip or internal channels of the endoscope.

Therefore, it is readily apparent that there is a recognizable unmet need for a distal tip soak basin, agitator, and methods of use thereof that functions to contain the distal tip and orifice in an enzymatic fluid for the purpose of pre-soaking and agitating the enzymatic fluid in the distal tip to delay the drying of body fluids and particulate contained inside the internal channels.

### BRIEF SUMMARY

Briefly described, in example embodiment, the present apparatus overcomes the above-mentioned disadvantage, and meets the recognized need for a distal tip soak basin, agitator, and methods of use, by providing a tube or sleeve with a closed end and an open end, an end cap having an aperture therein to retain a medical device, a perforated instrument holder or seal to seal the end cap around a medical device, a vibrating motor and hosing with friction fit sleeve, a battery, and an on/off switch, and, thus, functions to contain the distal tip and orifice of an endoscope in an enzymatic fluid for the purpose of pre-soaking and agitating the cleaning or soaking fluid, such as an enzymatic fluid, in the distal tip to delay the drying of body fluids and particulate contained inside the distal tip and orifice and/or internal channels.

According to its major aspects and broadly stated, the disclosure includes vibrating motor and hosing with friction fit sleeve, a battery, and an on/off switch affixed or removeably affixed to an open end tube with a sealed end and a cap and seal to pre-soaking and agitate a distal tip of an endoscope.

Furthermore, according to its major aspects and broadly stated, the distal tip soak basin, agitator, and methods of use includes a tubular sleeve with battery powered vibration motor and medical device retainer cap. The vibration motor is designed to slide over a plastic tube and the medical device retainer cap replaces the tube cap. After sliding the sleeve with vibration motor over the tube cleaning fluid is added to the tube and then the retainer cap is attached to the tube. The retainer cap has a sponge with a perforated opening designed to securely hold endoscopes and other complex medical devices. The disclosure allows for pre-soaking the distal tip of endoscopes and other complex medical devices with the addition of fluid agitation and damage protection provided by the hard plastic tube.

Accordingly, a feature of the distal tip soak basin, agitator, and methods of use is its ability to provide a sonic fluid agitation provided by the fluid agitation device or vibration motor attached to the distal tip soak basin.

In a preferred embodiment, an apparatus to agitate a soaking fluid therearound a distal tip of an endoscope having one or more channels therein, the apparatus includes an open-ended tube and an end cap, the end cap having a cap aperture, a sealing mechanism positioned between the end cap and the open-ended tube, and a vibration motor, the vibration motor having a housing therearound, wherein the housing includes a tube attachment collar, the tube attachment collar is configured to be affixed thereto the open-ended tube.

In still a further exemplary embodiment, a method of utilizing an apparatus to agitate a soaking fluid therearound a distal tip of an endoscope having one or more channels therein, the method including the steps of providing an open-ended tube and an end cap, the end cap having a cap aperture, a sealing mechanism positioned between the end cap and the open-ended tube, a vibration motor, the vibration motor having a housing therearound, wherein the housing includes a tube attachment collar, the tube attachment collar is configured to be affixed thereto the open-ended tube, inserting the distal tip of the endoscope therein the cap aperture of the end cap and one or more sealing mechanisms, sealing the distal tip of the endoscope therein the end cap and the tube submerged the a soaking fluid, activating the vibration motor; and agitating the cleaning fluid therearound the distal tip and therein the one or more internal conduits.

Accordingly, a feature of the distal tip soak basin, agitator, and methods of use is its ability to provide pre-soaking of the distal tip, bending section and internal channels of an endoscope.

Another feature of the distal tip soak basin, agitator, and methods of use is its ability to reduce or prevent damage to the distal tip and bending section of an endoscope. Cost reduction by preventing distal tip damage.

Still another feature of the distal tip soak basin, agitator, and methods of use is its ability to reduce the amount of cleaning or soaking fluid, such as an enzymatic fluid needed provided by the contoured design of distal tip basin.

Yet another feature of the distal tip soak basin, agitator, and methods of use is its ability to provide a leak proof way to flush instrument channels.

Yet another feature of the distal tip soak basin, agitator, and methods of use is its ability to provide a battery powered vibration motor for fluid agitation.

Yet another feature of the distal tip soak basin, agitator, and methods of use is its ability to prevent organic matter from drying on the distal tip of endoscope.

Yet another feature of the distal tip soak basin, agitator, and methods of use is its ability to provide sonic fluid agitation to assist with removal of bioburden accumulated on distal tip and internal channels of endoscopes.

Yet another feature of the distal tip soak basin, agitator, and methods of use is its ability to provide sonic fluid agitation to assist with removal of impacted bioburden commonly found on the elevator of duodenoscope and other complex endoscope distal tips.

Yet another feature of the distal tip soak basin, agitator, and methods of use is its ability to provide agitating fluid to assist with removal of organic matter from distal tip of endoscope. Sonic fluid agitation provided by the fluid agitation device attached to the distal tip soak basin.

Yet another feature of the distal tip soak basin, agitator, and methods of use is its ability to provide vibration frequency which resonates/transmits through endoscope channels and assist with removal organic matter during channel brushing.

Yet another feature of the distal tip soak basin, agitator, and methods of use is its ability to provide damage or impact protection of the distal tip by covering with a hard plastic lab tube.

Yet another feature of the distal tip soak basin, agitator, and methods of use is its ability to provide information tracking provided by a barcode label attached to the tube. Endoscope tracking and record keeping is captured by scanning the pre-printed barcode located on the distal tip soak basin. The tracking system captures reprocessing data to help ensure reprocessing protocols are followed and endoscopes are reprocessed as efficiently as possible.

Yet another feature of the distal tip soak basin, agitator, and methods of use is its ability to capture fluid injected through channels of the endoscope for the purpose of quality testing.

Yet another feature of the distal tip soak basin, agitator, and methods of use is its ability to provide an airtight basin and when filled with a soaking fluid a pre-leak test of the endoscope can be performed. The purpose of pre-leak testing is to detect holes in the bending section before the manual cleaning process begins. Early detection can help reduce fluid invasion which will help lower repair cost.

Yet another feature of the distal tip soak basin, agitator, and methods of use is its ability to provide an enhanced pre-cleaning cycle by presoaking and fluid agitation at point of use.

Yet another feature of the distal tip soak basin, agitator, and methods of use is its ability to provide additional pre-cleaning time during transport from the procedure room to the decontamination area. This is accomplished by keeping the distal tip and internal channels soaked and agitated throughout the transportation period.

Yet another feature of the distal tip soak basin, agitator, and methods of use is its ability to provide immediate pre-cleaning at point of use.

Yet another feature of the distal tip soak basin, agitator, and methods of use is its ability to continuously cleans throughout transportation to decontamination.

Yet another feature of the distal tip soak basin, agitator, and methods of use is its ability to reduce the likelihood of dried bioburden and biofilm formation.

Yet another feature of the distal tip soak basin, agitator, and methods of use is its ability to provide positive air pressure through the internal channels of endoscopes. This provides a simple way to dry the internal channels (retrograde filtered air for 48 hours) and protect the distal tip during storage.

Yet another feature of the distal tip soak basin, agitator, and methods of use is its ability to encapsulate the entire bending section of flexible endoscopes, which account for more then 75% of damage and reduces the likelihood of the distal tip hitting hard surfaces and being damaged during transport.

Yet another feature of the distal tip soak basin, agitator, and methods of use is its ability to enable digital tracking via bar or QR code labeling of the tube attached to endoscopes. The system enables tracking from pre-cleaning to storage and records all information suggested by Joint Commission and the FDA.

These and other features of the distal tip soak basin, agitator, and methods of use will become more apparent to one skilled in the art from the following Detailed Description of the Embodiments and Claims when read in light of the accompanying drawing Figures.

### BRIEF DESCRIPTION OF THE DRAWINGS

The present distal tip soak basin, agitator, and methods of use will be better understood by reading the Detailed Description of the embodiments with reference to the accompanying drawing figures, in which like reference numerals denote similar structure and refer to like elements throughout, and in which:

FIG. 1 is a front perspective view of an exemplary embodiment of an endoscope tip cover;

FIG. 2 is a front perspective view of an alternate exemplary embodiment of an endoscope tip cover;

FIG. 3 is a front perspective view of an exemplary distal tip of an endoscope;

FIG. 4 is a perspective view of an exemplary embodiment of a vibration motor and tube collar;

FIG. 5 is schematic diagram of the embodiment of FIG. 4;

FIG. 6 is a perspective view of the assembly of embodiments of FIGS. 1, 3 and 4;

FIG. 7 is a perspective view of an alternate exemplary embodiment of the vibration motor and tube collar of FIG. 4;

FIG. 8 is a perspective view of an exemplary embodiment of the end cap of FIGS. 1 and 2; and

FIG. 9 is a flow diagram of a method sport racket score keeping and vibration dampening.

It is to be noted that the drawings presented are intended solely for the purpose of illustration and that they are, therefore, neither desired nor intended to limit the disclosure to any or all of the exact details of construction shown, except insofar as they may be deemed essential to the claimed invention.

### DETAILED DESCRIPTION

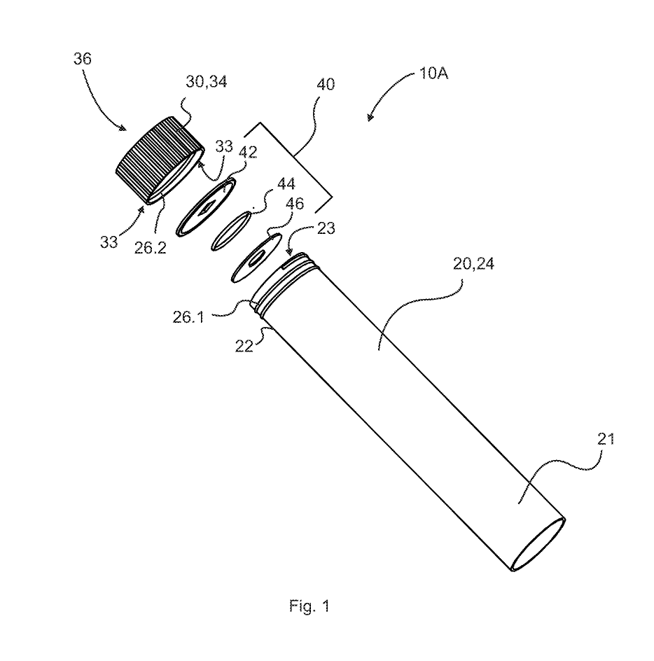

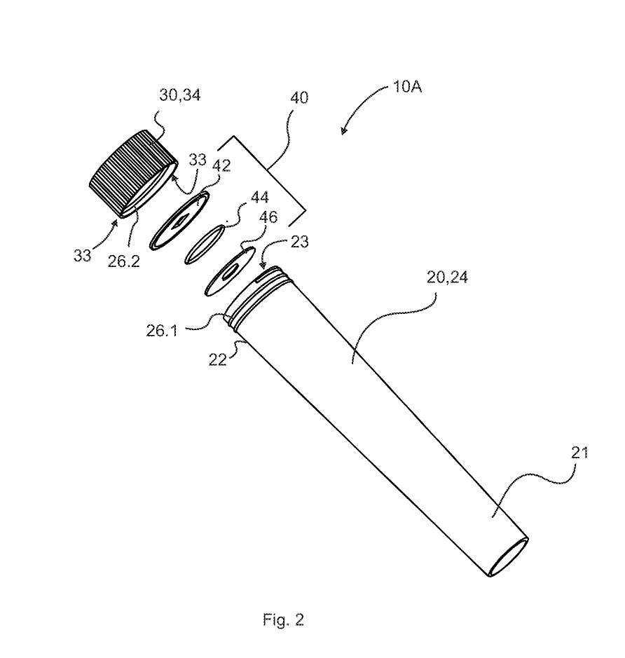

In describing the exemplary embodiments of the present disclosure, as illustrated in FIGS. 1-9, specific terminology is employed for the sake of clarity. The present disclosure, however, is not intended to be limited to the specific terminology so selected, and it is to be understood that each specific element includes all technical equivalents that operate in a similar manner to accomplish similar functions. Embodiments of the claims may, however, be embodied in many different forms and should not be construed to be limited to the embodiments set forth herein. The examples set forth herein are non-limiting examples, and are merely examples among other possible examples.

Referring now to FIGS. 1, 2, and 8, by way of example, and not limitation, there is illustrated an example embodiment apparatus of endoscope tip cover 10. Endoscope tip cover 10 may include an open-ended container, such as tube 20, having first tube end 21 and second tube end 22, wherein first tube end 21 is preferably sealed and second tube end 22 is preferably open or open-ended. Moreover, tube 20 may include interior tube surface 23 and exterior tube surface 24, wherein exterior surface 24 may include attachment device 26, such as second set of threads 26.1 formed therein exterior surface 24 and proximate second tube end 22. Endoscope tip cover 10 further includes end cap 30, wherein end cap 30 may include interior cap surface 33 and exterior cap surface 34, wherein interior cap surface 33 may include attachment device 26, such as first set of threads 26.2 formed therein interior surface 33. Preferably, attachment device 26, such as second set of threads 26.1 formed therein exterior surface 24 and proximate second tube end 22 and first set of threads 26.2 formed therein interior surface 33 may rotational engage one another to seal end cap 30 thereto second tube end 22 of tube 20. Furthermore, end cap 30 may include a hole, such as cap aperture 35.1 therethrough top surface 36, from exterior cap surface 34 to interior cap surface 33 to accommodate end section ES of linear member LM, such as a medical instrument, and more specifically an endoscope.

End cap 30 and second tube end 22 of tube 20 may include a perforated instrument holder, such as one or more sealing mechanism 40 to seal end section ES of linear member LM therein tube 20. One or more sealing mechanism 40 may include a flexible membrane seal, such as instrument port seal 42, wherein instrument port seal 42 preferably includes a hole, such as instrument port aperture 35.2 therethrough instrument port seal 42 to seal section ES of linear member LM therein end cap 30 and second tube end 22. One or more sealing mechanism 40 may include o-ring 44, wherein o-ring 44 preferably includes a hole, such as o-ring aperture 35.3 therethrough o-ring 44 to seal section ES of linear member LM therein end cap 30 and second tube end 22. One or more sealing mechanism 40 may include retaining washer 46, wherein retaining washer 46 preferably includes a hole, such as retaining washer aperture 35.4 therethrough retaining washer 46 to seal section ES of linear member LM therein end cap 30 and second tube end 22.

It is contemplated herein that attachment device 26 may include a friction fitting, snap fitting, or other sealing means known by one of ordinary skill in the art.

It is contemplated herein that end cap 30 and tube 20 may be preferably made of one or more of a variety of shock absorbing or impact resistant materials whether rigid, semi-rigid or flexible, including by way of example but not by way of limitation, plastic, rubber, acrylic, foam rubber, or a rubber-like material or other impact resistant material ideal for enabling end cap 30 and tube 20 to protect section ES of linear member LM.

It is further contemplated herein that instrument port seal 42, o-ring 44, and retaining washer 46 may be preferably made of one or more of a variety of flexible materials whether rigid, semi-rigid or flexible, including by way of example but not by way of limitation, plastic, rubber, foam rubber, or a rubber-like material or other sealant material ideal for enabling instrument port seal 42, o-ring 44, and retaining washer 46 to seal section ES of linear member LM therein end cap 30 and second tube end 22.

It is still further contemplated herein that instrument port seal 42, o-ring 44, and retaining washer 46 may be configured from a sponge material with a perforated opening, such as instrument port seal 42 designed to securely hold and seal linear member LM (including endoscope and other complex medical devices) therein end cap 30 and second tube end 22 and other complex medical devices.

It is still further contemplated herein that tube 20 may be configured to accommodate linear member LM, such as, tube 20 may tapper or narrow from second tube end 22 to first tube end 21.

It is still further contemplated herein that tube 20 and vibration motor 70 may be separate pieces or may be integral wherein exterior surface 24 of tube 20 is formed integral thereto housing exterior surface 76 of motor housing 72.

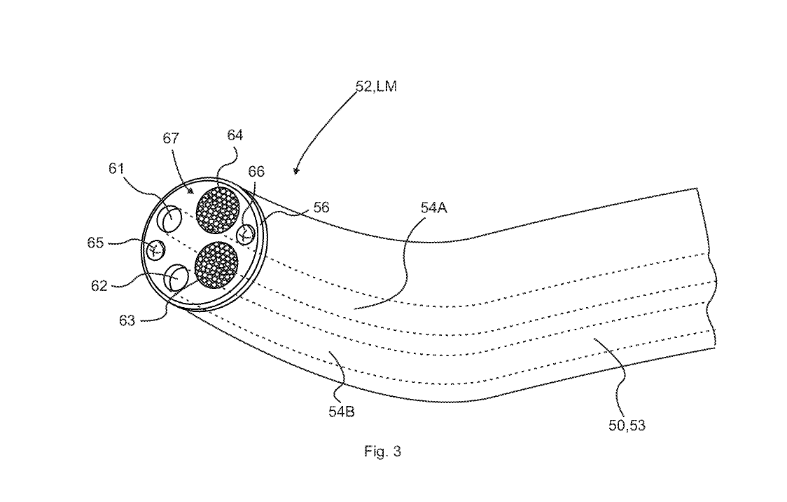

Referring now to FIG. 3, by way of example, and not limitation, there is illustrated an example embodiment of distal tip 52 of an endoscope 50. Distal tip 52 of an endoscope 50 may include one or more internal channels or passageways, such as conduit 54 traversing internally the length of flexible bending section 53. One or more internal conduits 54 such as first conduit 54A and second conduit 54B, which may include conduits for delivery of water via pressurized water conduit 61 or delivery of air via air or compressed air conduit 62, fiber optic image bundle or image capture sensor via conduit 63, fiber optic lights or image capture sensor via conduit 64, delivery of vacuum via suction conduit 65, delivery of medical instrument, such as a suture to conduct a biopsy via medical instrument conduit 66 and like conduits to meet the needs of the medical application being performed. Moreover, one or more internal conduits 54 may be covered by a flexible sheath 56 to protect distal tip 52 of an endoscope 50.

It is known by one of ordinary skill in the art that disinfection of endoscope 50 is difficult due to the miniature design of the endoscope 50, hidden and/or retractable instruments, small orifices, and long narrow channels or passageways, such as include conduits for delivery of water via pressurized water conduit 61 or delivery of air via air or compressed air conduit 62, fiber optic image bundle or image sensor or image capture conduit 63, fiber optic bundle or light source conduit 64, delivery of vacuum via suction conduit 65, delivery of medical instrument, such as a suture to conduct a biopsy via medical instrument conduit 66 and like. To prevent body fluids and particulate (bioburden 100) from drying in these remote locations after use a pre-cleaning apparatus, such as endoscope tip cover 10 may be utilized.

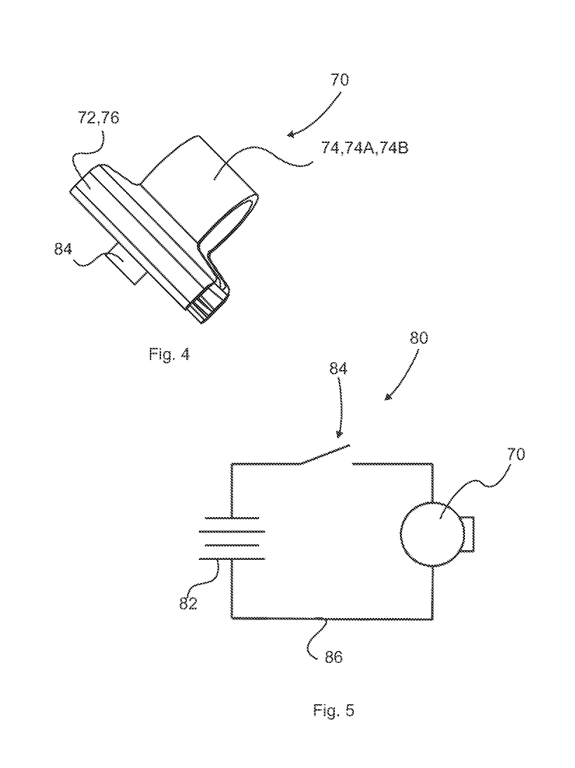

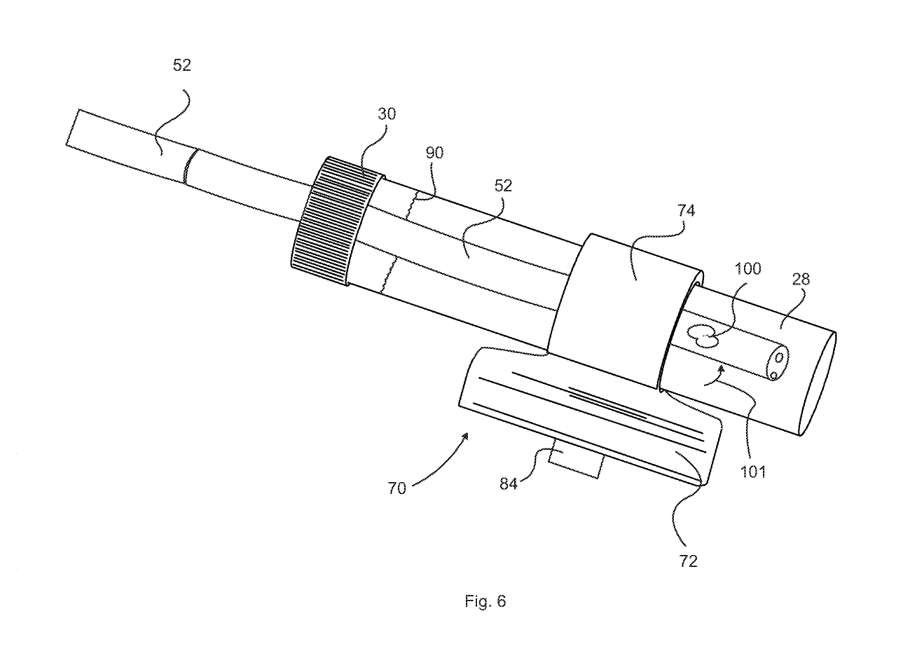

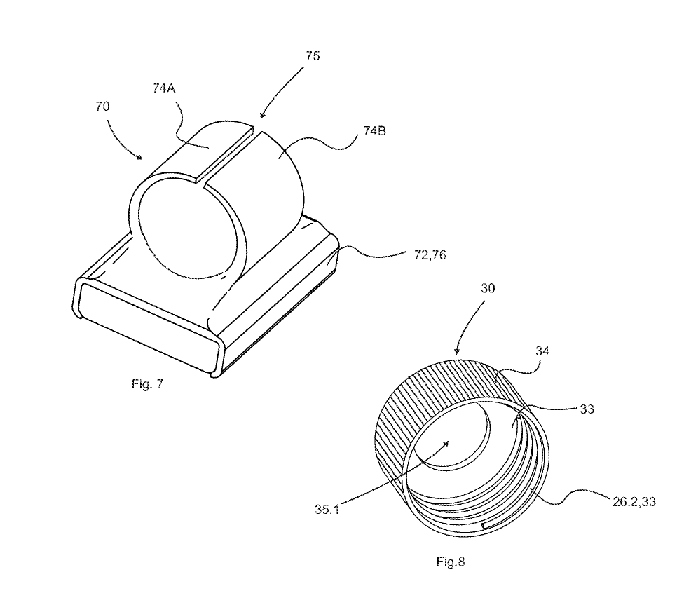

Referring now to FIGS. 4 and 7, by way of example, and not limitation, there is illustrated an example embodiment of an apparatus for cleaning distal tip 52 of endoscope 50 via agitation or vibration device, such as vibration motor 70. Vibration motor 70 includes motor housing 72 wherein motor housing 72 may further include a tubular sleeve, such as tube attachment collar 74 formed therein or affixed thereto housing exterior surface 76 of motor housing 72. Tube attachment collar 74 is preferably designed to slide over exterior surface 24 of tube 20 or releasably affixed thereto exterior surface 24 of tube 20 and, thus releasably attach motor housing 72 to tube 20, as shown in FIG. 6. Moreover, motor housing 72 may be releasably affixed thereto exterior surface 24 of tube 20 by other means such as friction fit, threaded, glued, hook and loop, groove and key, or the like known to one of ordinary skill in the art.

It is contemplated herein that motor housing 72 may be formed integral thereto tube 20 rather than formed as a separate component to be releasably affixed thereto tube 20.

It is further contemplated herein that tube attachment collar 74 may be split into first collar section 74A and second collar section 74B having gap 75 therebetween to enable tube attachment collar 74 to flex and accommodate different diameter or sized tube 20.

Referring now to FIG. 5, by way of example, and not limitation, there is illustrated an example embodiment of a schematic circuit diagram 80 of agitation or vibration device, such as vibration motor 70. Vibration motor 70 preferably includes a power supply, such as battery 82, electrically connected (one or more electrical connection 86) thereto on/off/speed switch 84, electrically connected thereto vibration motor 70, which is preferably electrically connected thereto battery 82. It is contemplated herein that circuitry and/or electrically connections may further include variable speed motor, rheostat, and other resistor, capacitor, inductor, and the like 70 necessary to achieve necessary agitation or vibration from vibration motor 70.

Referring again to FIGS. 1, 2, 4, and 6, by way of example, and not limitation, there is illustrated an example embodiment of vibration motor 70. In use, distal tip 52 of endoscope 50 is preferably inserted therethrough cap aperture 35.1 of end cap 30 and one or more sealing mechanism 40, such as instrument port aperture 35.2 of instrument port seal 42, o-ring aperture 35.3 of o-ring 44, retaining washer aperture 35.4 of retaining washer 46, wherein distal tip 52 of endoscope 50 may be sealed therein end cap 30 and tube 20 submerged in cleaning fluid, such as enzymatic fluid 90. Vibration motor 70 may be releasably affixed to tube 20 by inserting tube 20 therein tube attachment collar 74 of housing exterior surface 76 of motor housing 72. Vibration motor 70 may be activated by changing the state of on/off/speed switch 84 whereby vibration motor 70 vibrates or agitates (vibration) motor housing 72, attachment collar 74, tube 20, cleaning fluid, such as enzymatic fluid 90 and distal tip 52 of endoscope 50. It is recognized herein that vibration or agitation via vibration motor 70 creates eddy currents of fluid flow 101 or fluid dynamics within cleaning fluid, such as enzymatic fluid 90 therein tube 20, which flow around distal tip 52 of endoscope 50 and in and out of one or more internal conduits 54, such as, first conduit 54A and second conduit 54B, pressurized water conduit 61, compressed air conduit 62, image capture conduit 63, light source conduit 64, suction conduit 65, and medical instrument conduit 66 to prevent body fluids and particulate (bioburden 100) from drying in these areas from post operation through transit to decontamination.

It is contemplated herein that cleaning fluid, such as enzymatic fluid 90 may be added to tube 20 prior to inserting distal tip 52 of endoscope 50 therein tube 20 or after distal tip 52 of endoscope 50 is inserted therein tube 20.

It is contemplated herein that vibration motor 70, end cap 30, one or more sealing mechanism 40, and tube 20 enable pre-soaking distal tip 52 of endoscope 50 and other complex medical devices with the addition of fluid agitation and damage protection provided by the hard plastic lab tube.

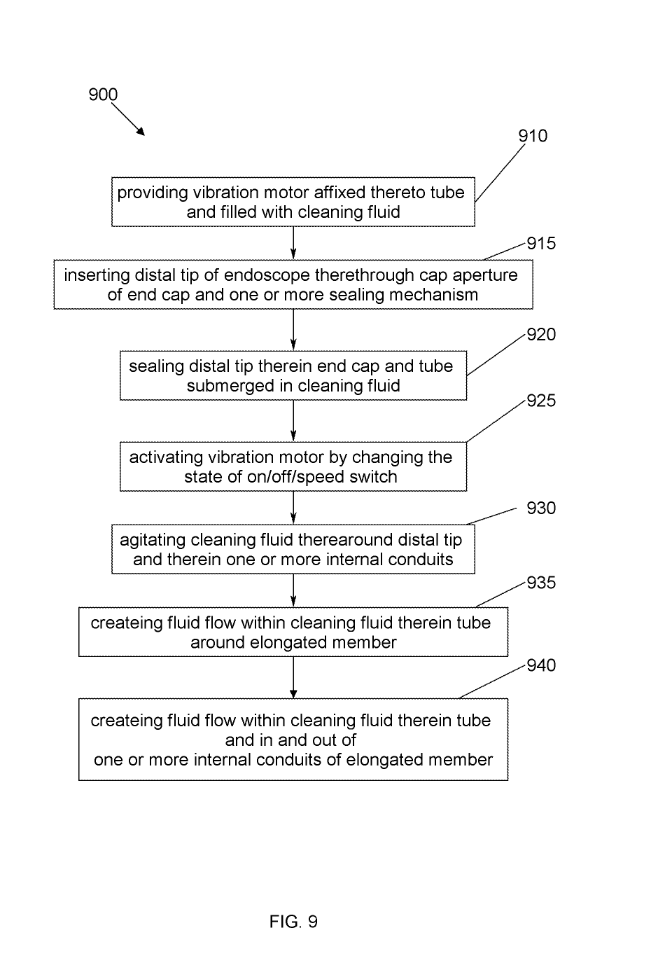

Referring now to FIGS. 6 and 9, there is illustrated a flow diagram 700 of utilizing vibration motor 70 to pre-soak and agitate or vibrate distal tip 52 of endoscope 50 in cleaning fluid, such as enzymatic fluid 90. In block or step 910, providing vibration motor 70, tube 20, one or more sealing mechanism 40, end cap 30 filled with cleaning fluid, such as enzymatic fluid 90, and assembly of tube 20 therein tube attachment collar 74 of vibration motor 70, and sliding tube attachment collar 74 over tube 20 or inserting tube 20 in tube attachment collar 74, as described above in FIGS. 1-6. In block or step 915, inserting distal tip 52 of endoscope 50 therethrough cap aperture 35.1 of end cap 30 and one or more sealing mechanism 40.

In block or step 920, sealing or inserting distal tip 52 of endoscope 50 therein tube 20, end cap 30, and one or more sealing mechanism 40, such as instrument port aperture 35.2 of instrument port seal 42, o-ring aperture 35.3 of o-ring 44, retaining washer aperture 35.4 of retaining washer 46, wherein distal tip 52 of endoscope 50 may be submerged in cleaning fluid, such as enzymatic fluid 90.

In block or step 925, activating vibration motor 70 by changing the state of on/off/speed switch 84 wherein vibration motor 70 creates eddy currents of fluid flow 101 or fluid dynamics within cleaning fluid, such as enzymatic fluid 90 therein tube 20, which flow around distal tip 52 of endoscope 50 and in and out of one or more internal conduits 54. In block or step 930, agitating cleaning fluid such as enzymatic fluid 90 therearound distal tip 52 of endoscope 50 and therein one or more internal conduits 54. In block or step 935, creating fluid flow 101 therein tube around distal tip 52 of endoscope 50 to prevent body fluids and particulate (bioburden 100) from drying in these areas from post operation through transit to decontamination. In block or step 940, creating fluid flow 101 therein tube 20 in and out of one or more internal conduits 54, such as, first conduit 54A and second conduit 54B, pressurized water conduit 61, compressed air conduit 62, image capture conduit 63, light source conduit 64, suction conduit 65, and medical instrument conduit 66 to prevent body fluids and particulate (bioburden 100) from drying in these areas from post operation through transit to decontamination.

The foregoing description and drawings comprise illustrative embodiments of the present disclosure. Having thus described exemplary embodiments, it should be noted by those ordinarily skilled in the art that the within disclosures are exemplary only, and that various other alternatives, adaptations, and modifications may be made within the scope of the present disclosure. Merely listing or numbering the steps of a method in a certain order does not constitute any limitation on the order of the steps of that method. Many modifications and other embodiments of the disclosure will come to mind to one ordinarily skilled in the art to which this disclosure pertains having the benefit of the teachings presented in the foregoing descriptions and the associated drawings. Although specific terms may be employed herein, they are used in a generic and descriptive sense only and not for purposes of limitation. Moreover, the present disclosure has been described in detail, it should be understood that various changes, substitutions and alterations can be made thereto without departing from the spirit and scope of the disclosure as defined by the appended claims. Accordingly, the present disclosure is not limited to the specific embodiments illustrated herein, but is limited only by the following claims.

What is claimed is:

1. An apparatus to agitate a cleaning fluid therearound a distal tip of an endoscope having one or more channels therein, said apparatus comprising:
an open-ended tube and an end cap, said end cap having a cap aperture;
a sealing mechanism positioned between said end cap and said open ended tube; and
a vibration motor, said vibration motor having a housing therearound, wherein said housing having a tube attachment collar, said tube attachment collar is configured to be affixed thereto said open-ended tube.

2. The apparatus of claim 1, wherein said vibration motor further comprises a power supply electrically connected to a vibration motor.

3. The apparatus of claim 2, wherein said vibration motor further comprises an on/off switch electrically connected between said power supply electrically and said vibration motor.

4. The apparatus of claim 2, wherein said power supply further comprises a battery.

5. The apparatus of claim 2, wherein said vibration motor further comprises a variable speed motor.

6. The apparatus of claim 5, wherein said vibration motor further comprises a rheostat electrically connected between said power supply electrically and said vibration motor.

7. The apparatus of claim 5, wherein said end cap further comprises a top surface for said cap aperture and an interior surface, wherein said interior surface having an attachment device to releasably affix said cap to said open-ended tube.

8. The apparatus of claim 7, wherein said attachment device further comprises a first set of threads.

9. The apparatus of claim 7, wherein said open-ended tube further comprises an exterior surface, said exterior surface further comprises a second set of threads.

10. The apparatus of claim 7, wherein said sealing mechanism further comprises an instrument port seal.

11. The apparatus of claim 7, wherein said sealing mechanism further comprises an o-ring.

12. The apparatus of claim 7, wherein said sealing mechanism further comprises a retaining washer.

13. The apparatus of claim 1, wherein said tube attachment collar is configured to be releasably affixed thereto said open-ended tube.

14. The apparatus of claim 1, wherein said tube attachment collar further comprises a first collar section and a second collar section having a gap therebetween to accommodate different sized said open-ended tube.

15. The apparatus of claim 1, wherein the cleaning fluid further comprises an enzymatic fluid.

16. A method of utilizing an apparatus to agitate a cleaning fluid therearound a distal tip of an endoscope having one or more channels therein, said method comprising the steps of:
providing an open-ended tube and an end cap, said end cap having a cap aperture, a sealing mechanism positioned between said end cap and said open-ended tube, a vibration motor, said vibration motor having a housing therearound, wherein said housing having a tube attachment collar, said tube attachment collar is configured to be affixed thereto said open-ended tube;
inserting the distal tip of the endoscope therein said cap aperture of said end cap and one or more sealing mechanisms;
sealing the distal tip of the endoscope therein said end cap and said tube submerged in the cleaning fluid;
activating said vibration motor; and
agitating the cleaning fluid therearound the distal tip and therein the one or more internal conduits.

17. The method of claim 16, further comprises the step of creating a fluid flow therein said open-ended tube and around the distal tip of the endoscope to prevent a bioburden from drying thereon the distal tip.

18. The method of claim 16, further comprises the step of creating a fluid flow therein said open-ended tube and therein one or more internal conduits of the distal tip of the endoscope to prevent a bioburden from drying therein said one or more internal conduits.
